(19) United States
(12) Patent Application Publication
(10) Pub. No.: US 2006/0089586 A1
(43) Pub. Date: Apr. 27, 2006

(54) Convertible extracorporeal blood perfusion systems

(76) Inventors: Stanley B. Kaus, Longmont, CO (US); Thomas A. Rawles, Plano, TX (US); Anthony Kenneth Litzie, Tustin Ranch, CA (US)

Correspondence Address: POPOVICH, WILES & O'CONNELL, PA;650 THIRD AVENUE SOUTH, SUITE 600, MINNEAPOLIS, MN, 55402, US

(21) Appl. No.: 11/254,227

(22) Filed: Oct. 19, 2005

Related U.S. Application Data
(60) Provisional application No. 60/621,294, filed on Oct. 22, 2004.

(51) Int. Cl. A61M 37/00

(52) U.S. Cl. 604/4.01; 604/6.14

(57) ABSTRACT

A convertible extracorporeal blood perfusion system for receiving venous blood from a patient and for returning oxygenated blood to the patient in a cardiopulmonary bypass procedure including: a closed-loop cardiopulmonary bypass system; and a circuit for converting between the closed-loop cardiopulmonary bypass system and a cardiopulmonary bypass system containing a venous reservoir, the circuit for converting comprising a venous reservoir that can be fluidly connected to a venous line from the patient and that can be fluidly connected to the inlet of a first pump, the first pump being part of the closed-loop cardiopulmonary bypass system, the closed loop cardiopulmonary bypass system comprising a bubble removal device fluidly connected to the inlet of the first pump.

This application claims the benefit of U.S. Provisional Patent Application No. 60/621,294, filed Oct. 22, 2004, the contents of which are hereby incorporated herein by reference.

### FIELD OF THE INVENTION

This invention relates to extracorporeal blood perfusion systems or cardiopulmonary bypass (“CPB”) systems that pump, oxygenate and may filter blood to maintain patient viability during cardiopulmonary bypass procedures and other procedures requiring circulatory and/or respiratory patient support, all such procedures henceforth to be cumulatively and inclusively, but not strictly referred to as cardiopulmonary bypass, or CPB. More particularly, the invention relates to a convertible cardiopulmonary blood perfusion bypass system that allows ready conversion between a closed-loop cardiopulmonary bypass mode and a cardiopulmonary bypass mode that includes a venous reservoir.

### BACKGROUND OF THE INVENTION

Extracorporeal blood perfusion systems used for cardiopulmonary bypass typically consist of a hardshell venous reservoir or venous reservoir bag, a peristaltic or centrifugal blood pump, a heat exchanger, an oxygenator, and an arterial filter. Venous reservoirs are used in CPB systems to provide capacitance to the extracorporeal system and blood volume storage capability as well as to provide means for air removal and blood filtration.

Closed-loop cardiopulmonary bypass systems are intended to be operated without an obligatory venous reservoir. In general, closed-loop cardiopulmonary bypass systems are characterized by direct kinetic assisted venous drainage using a centrifugal blood pump. In most closed-loop CPB systems presently available, a venous bubble trap placed in the venous line between the patient and the centrifugal blood pump captures air that may be present in the venous line and prevents this air from circulating through the cardiopulmonary system and back to the patient. In contrast to a traditional cardiopulmonary bypass system with a hardshell venous reservoir that passively removes venous air using the buoyancy of air in a liquid, silicone oil/silica particle defoaming agents, and the presence of a direct air/blood interface, a closed-loop cardiopulmonary bypass system requires an active air removal system. A purge line attached to the bubble trap can be used to actively remove the accumulation of air.

Closed-loop cardiopulmonary bypass systems offer a number of features that may not be available with typical cardiopulmonary bypass systems containing a hardshell venous reservoir or venous reservoir bag. These features include: (1) a reduction in hemodilution accomplished by a reduction in extracorporeal prime volume; (2) a reduction in blood component damage and/or activation accomplished by reduction of non-endothelial foreign surface area and minimization of air/blood interface; and (3) a reduction in particulate embolism accomplished by minimization of exposure of the patient's blood to silicone/silica eluting defoaming agents.

The convertible extracorporeal blood perfusion system of the invention can be used with any closed-loop CPB system or any integrated cardiopulmonary mini-bypass device. Integrated cardiopulmonary mini-bypass devices that are currently known include the COBE SYNERGY Adult Integrated Mini Bypass System and the CARDIOVENTION CORX System. Closed-loop CPB systems that are currently known include the COBE SYNERGY Adult Integrated Mini Bypass System, the CARDIOVENTION CORX System, the MEDTRONIC RESTING HEART System, and the NOVOSCI READY System.

One of the significant challenges of closed-loop cardiopulmonary bypass systems involves the management of significant volumes of air in the venous blood that may occur during CPB procedures. With the convertible bypass system of this disclosure, the benefits of a closed-loop cardiopulmonary bypass system can be achieved with the advantages of a readily available hardshell venous reservoir or venous reservoir bag.

### SUMMARY OF THE INVENTION

The invention provides a convertible extracorporeal blood perfusion system for receiving venous blood from a patient and for returning oxygenated blood to the patient in a cardiopulmonary bypass procedure comprising: a closed-loop cardiopulmonary bypass system; and a circuit for converting between the closed-loop cardiopulmonary bypass system and a cardiopulmonary bypass system containing a venous reservoir, the circuit for converting comprising a venous reservoir that can be fluidly connected to a venous line from the patient and that can be fluidly connected to the inlet of a first pump, the first pump being part of the closed-loop cardiopulmonary bypass system, the closed-loop cardiopulmonary bypass system comprising a bubble removal device fluidly connected to the inlet of the first pump. The invention also provides a method for converting the convertible extracorporeal blood perfusion system between a closed-loop cardiopulmonary bypass system and a cardiopulmonary bypass system containing a venous reservoir, comprising fluidly connecting the venous reservoir to the venous line from the patient and fluidly connecting the venous reservoir to the inlet of the first pump.

It is to be understood that both the foregoing general description and the following detailed description are exemplary and explanatory and are intended to provide further explanation of the invention as claimed.

### BRIEF DESCRIPTION OF THE DRAWINGS

FIG. 1 shows a convertible extracorporeal blood perfusion system of the invention with a venous reservoir bag for use with a mini-bypass device.

FIG. 2 illustrates an integrated cardiopulmonary mini-bypass device that includes a venous bubble trap, centrifugal pump, oxygenator/heat exchanger, and arterial filter.

FIG. 3A illustrates a convertible extracorporeal blood perfusion system of the invention with a hardshell venous reservoir for use with a mini-bypass device.

FIG. 3B is an illustration similar to that of FIG. 3A, showing the positioning of specialized sensors and control devices.

### DESCRIPTION OF THE PREFERRED EMBODIMENTS

The invention provides a convertible extracorporeal blood perfusion system for receiving venous blood from a patient and for returning oxygenated blood to the patient in a cardiopulmonary bypass procedure comprising: a closed-loop cardiopulmonary bypass system; and a circuit for converting between the closed-loop cardiopulmonary bypass system and a cardiopulmonary bypass system containing a venous reservoir, the circuit for converting comprising a venous reservoir that can be fluidly connected to a venous line from the patient and that can be fluidly connected to the inlet of a first pump, the first pump being part of the closed-loop cardiopulmonary bypass system, the closed-loop cardiopulmonary bypass system comprising a bubble removal device fluidly connected to the inlet of the first pump. In the context of this invention, the terms “bubble trap” and “bubble removal device” are used in the specification and claims to mean broadly any air removal technology or device.

In another embodiment, the convertible extracorporeal blood perfusion system includes a bubble sensor on the venous line from the patient that is operable to send a signal when gaseous bubbles are detected to a controller that automatically activates a second pump fluidly attached to the bubble removal device in order to purge air from the bubble removal device and venous line. In another embodiment, the convertible extracorporeal blood perfusion system includes a bubble sensor on a first pump inlet line to the inlet of the first pump that is operable to send a signal when bubbles are detected in the first pump inlet line to a controller that automatically closes a clamp on an arterial line to the patient in order to stop the flow of blood back to the patient.

In one embodiment, the convertible extracorporeal blood perfusion system includes a level sensor that is operable to detect a blood level in the venous reservoir that is below a predetermined level and is operable to send a signal to a controller that automatically stops the flow of blood from the venous reservoir. In one embodiment, the controller automatically stops the flow of blood from the venous reservoir by closing a clamp on a venous reservoir exit line. In another embodiment, the controller automatically stops the flow of blood from the venous reservoir by stopping a second pump on a venous reservoir exit line. In another embodiment, the controller automatically stops the flow of blood from the venous reservoir by closing a clamp on an arterial line to the patient. In another embodiment, the convertible extracorporeal blood perfusion system includes a level sensor that is operable to detect a blood level in the venous reservoir that is below a predetermined level and is operable to send a signal to a controller that automatically closes a clamp on an arterial line to the patient in order to stop the flow of blood to the patient.

In one embodiment, the convertible extracorporeal blood perfusion system includes a level sensor that is operable to detect a blood level in the venous reservoir that is above a predetermined level and is operable to send a signal to a controller that automatically starts the flow of blood from the venous reservoir. In one embodiment, the controller automatically starts the flow of blood from the venous reservoir by opening a clamp on a venous reservoir exit line. In another embodiment, the controller automatically starts the flow of blood from the venous reservoir by starting a second pump on a venous reservoir exit line.

In one embodiment, the venous reservoir is a soft bag venous reservoir. In another embodiment, the soft bag venous reservoir is capable of vacuum assisted venous drainage. In another embodiment, the venous reservoir is a hardshell venous reservoir. In another embodiment the hardshell venous reservoir is capable of vacuum-assisted venous drainage.

In one embodiment, the convertible extracorporeal perfusion system contains a tubing segment that directly connects to the arterial and venous lines forming an A/V bridge. In another embodiment the outlet of the venous reservoir connects directly to the A/V bridge allowing for volume to be shifted from the patient to the reservoir or from the reservoir to the patient when operated in the closed-loop mode. In another embodiment, the venous reservoir is capable of filtering cardiotomy blood returned from the surgical site and returning this blood to the cardiopulmonary circuit or sequestering this blood for autologous blood salvage. In another embodiment, a tubing segment is connected to a filtered cardiotomy port on the venous reservoir to allow for homologous blood transfusions.

In one embodiment, a separate cardiotomy reservoir is used to filter cardiotomy blood returned from the surgical site that can be returned to the cardiopulmonary circuit or sequestered for autologous blood salvage.

In one embodiment, one or more prime bags are connected directly to the A/V bridge allowing for simple priming of the circuit and for volume to be shifted from the patient to the prime bags or from the prime bags to the patient when operated in the closed loop mode. The circuit configured with the prime bags in this manner allows for the additional benefit of antegrade and retrograde autologous priming of the system. In another embodiment, a quick-lock adapter is provided to allow for one of the prime bags to be easily replaced with a dedicated blood storage bag that allows for blood volume to be shifted from the patient to the blood storage bag or from the blood storage bag to the patient. One advantage provided by this blood storage bag is the elimination of exposure of this stored blood to silicone/silica eluting defoaming agents, minimization of exposure to non-endothelial foreign surface area and minimization of air/blood interface.

In one embodiment, the closed-loop cardiopulmonary bypass system further comprises an oxygenator and a heat exchanger. In another embodiment, the bubble removal device, first pump, oxygenator, and heat exchanger are integrated in one unit. In another embodiment, the closed-loop cardiopulmonary bypass system further comprises an oxygenator, a heat exchanger, and an arterial filter. In another embodiment, the bubble removal device, first pump, oxygenator, heat exchanger, and an arterial filter are integrated in one unit.

The invention provides a convertible extracorporeal blood perfusion system for receiving venous blood from a patient and for returning oxygenated blood to the patient in a cardiopulmonary bypass procedure comprising: a closed-loop cardiopulmonary bypass system; and a circuit for converting the closed-loop cardiopulmonary bypass system to a cardiopulmonary bypass system containing a venous reservoir, the circuit for converting comprising a venous reservoir that can be fluidly connected to a venous line from the patient and that can be fluidly connected to the inlet of a first pump, the first pump being part of the closed-loop cardiopulmonary bypass system, the convertible extracorporeal blood perfusion system comprising a level sensor that is operable to detect a blood level in the venous reservoir that is below a predetermined level and is operable to send a signal to a controller that automatically stops the flow of blood from the venous reservoir. In one embodiment, the controller automatically stops the flow of blood from the venous reservoir by closing a clamp on a venous reservoir exit line. In another embodiment, the controller automatically stops the flow of blood from the venous reservoir by stopping a second pump on a venous reservoir exit line.

The invention provides a convertible extracorporeal blood perfusion system for receiving venous blood from a patient and for returning oxygenated blood to the patient in a cardiopulmonary bypass procedure comprising: a closed-loop cardiopulmonary bypass system; and a circuit for converting the closed-loop cardiopulmonary bypass system to a cardiopulmonary bypass system containing a venous reservoir, the circuit for converting comprising a venous reservoir that can be fluidly connected to a venous line from the patient and that can be fluidly connected to the inlet of a first pump, the first pump being part of the closed-loop cardiopulmonary bypass system, the convertible extracorporeal blood perfusion system comprising a level sensor that is operable to detect a blood level in the venous reservoir that is below a predetermined level and is operable to send a signal to a controller that automatically closes a clamp on an arterial line to the patient in order to stop the flow of blood to the patient.

The invention also provides a method for converting the convertible extracorporeal blood perfusion system between a closed-loop cardiopulmonary bypass system and a cardiopulmonary bypass system containing a venous reservoir, comprising fluidly connecting the venous reservoir to the venous line from the patient and fluidly connecting the venous reservoir to the inlet of the first pump.

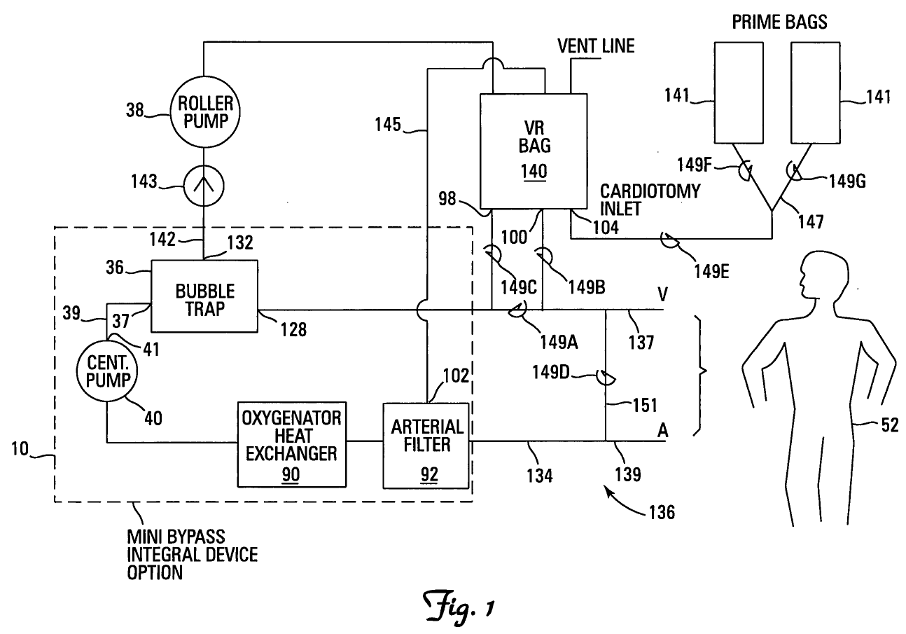

The present invention provides various extracorporeal blood perfusion systems that incorporate features and components that allow for the removal of air bubbles from the blood flowing through the system before the blood returns to the patient. FIG. 1 discloses a system that incorporates various components including a venous reservoir bag in a manner intended primarily for use as a closed-loop cardiopulmonary bypass system although, at the option of the user, provision is made to incorporate the venous reservoir bag into the system for use as a conventional gravity assisted or vacuum assisted venous drainage closed venous reservoir.

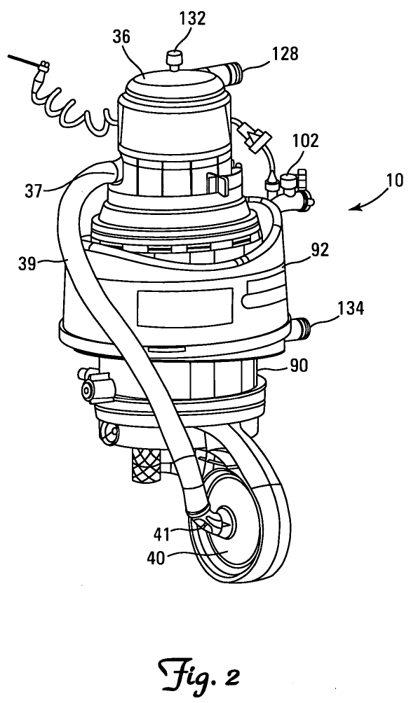

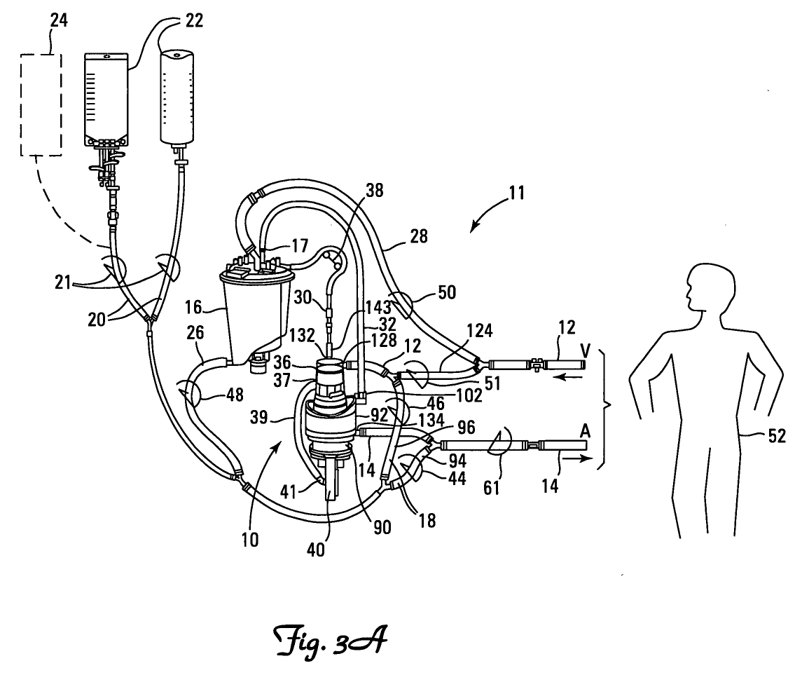

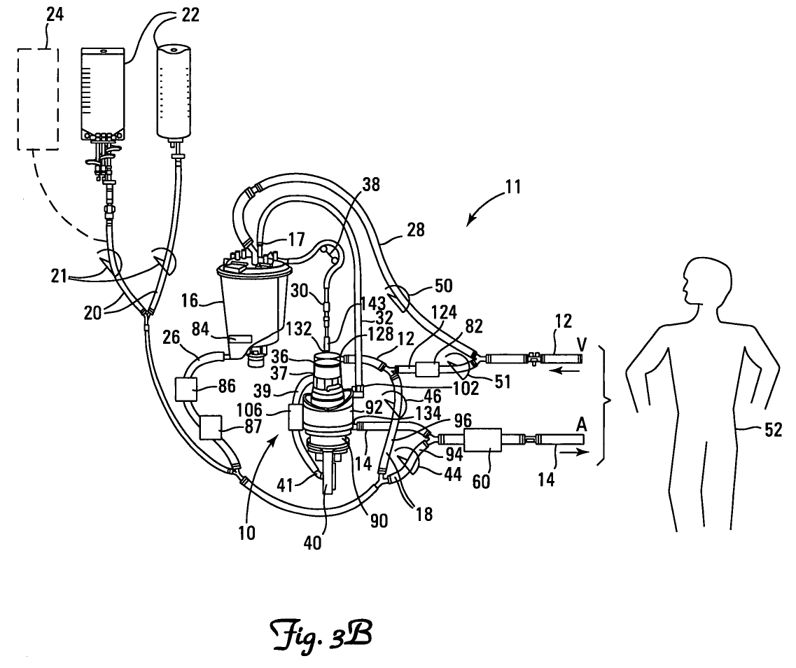

The extracorporeal systems shown in FIGS. 3A and 3B include a hardshell venous reservoir instead of a venous reservoir bag as in the system shown in FIG. 1. Certain other modifications and enhancements have been made including changes that allow the systems of FIGS. 3A and 3B, at the option of the user, to be quickly and safely converted between a closed-loop bypass mode and a conventional gravity assisted or vacuum assisted venous drainage hardshell reservoir mode. The perfusion system of FIG. 3B is similar to that of FIG. 3A except that certain hardware system control and safety features have been added. FIG. 2 shows an integrated mini-bypass device which may be used in the perfusion systems of FIGS. 1, 3A and 3B. A detailed description of the mini-bypass device and its use in these perfusion systems follows.

Integrated Cardiopulmonary Mini-Bypass Device

A mini-bypass device 10, which may be used in the novel extracorporeal blood perfusion systems disclosed herein, is shown in FIG. 2. Device 10 combines in a single unitary structure a blood oxygenator and heat exchanger 90, an arterial filter 92, a venous bubble trap 36 and a centrifugal pump 40. One example of such a mini-bypass device is shown and described in co-pending U.S. patent application Ser. No. 10/804,583, filed Mar. 18, 2004, entitled “Device and Methods for Processing Blood in Extracorporeal Circulation,” the contents of which are hereby incorporated herein by reference. Integrated mini-bypass devices presently available include the COBE SYNERGY Adult Integrated Mini Bypass System and the CARDIOVENTION CORX System.

Device 10 provides for extracorporeal circulation, oxygenation, filtration, and temperature control of a patient's blood during a CPB procedure. This mini-bypass device 10 is indicated for use in surgical procedures requiring extracorporeal cardiopulmonary support. Although device 10 may be a single integrated device that incorporates multiple components as shown in FIG. 2, the advantages of the present novel perfusion system are also achieved where some or all of the components of device 10 are provided as separately interconnected components, as shown in FIG. 1 which shows an extracorporeal blood perfusion system in which the mini-bypass device is used.

Convertible Cardiopulmonary Perfusion System Containing a Venous Reservoir Bag

A convertible extracorporeal cardiopulmonary perfusion system 136 is shown in FIG. 1. System 136 includes venous line 137, arterial line 139, the integrated mini-bypass device 10, one or more venous reservoir bags 140, one or more priming bags 141, priming lines 147, a bubble trap purge line 142 with a one-way valve 143, an arterial filter purge line 145 along with various interconnection tubing and line clamps 149A-G. As explained above, the device 10 includes blood oxygenator and heat exchanger 90, arterial filter 92, venous bubble trap 36 and centrifugal pump 40. The bubble trap purge line 142 is attached to bubble trap purge outlet 132. Line 39 connects bubble trap outlet 37 to centrifugal pump inlet 41. The system 136 may also include a blood gas sampling system, pressure monitoring lines, vent lines, suction lines, a cardiotomy reservoir, a cardioplegia line, and a gas line, not shown but as will be understood by those of skill in the art.

Extracorporeal cardiopulmonary perfusion system 136 provides an A/V loop that connects the mini-bypass device 10 to the patient 52 and provides the primary blood flow path during a CPB procedure. The venous line 137 directs blood flow from the patient 52 to the inlet 128 of the mini-bypass device 10. The arterial line 139 directs blood flow from the outlet 134 of the mini-bypass device 10 back to the patient 52. Although a venous reservoir bag 140 attaches to the venous line 137, the inlet and outlet to this reservoir bag 140 are normally closed (clamps 149B and 149C) and venous line clamp 149A is normally open so venous blood flow bypasses the reservoir bag 140 to flow directly into the mini-bypass device 10. In extracorporeal perfusion system 136, the A/V loop, includes an A/V bridge 151 that allows for recirculation through the device 10 with the patient's cardiovascular system temporarily clamped out of the system 136. This A/V bridge 151 and recirculation technique may be used to reprime the device 10 and the system 136 in the event that the mini-bypass device is deprimed. Fluid volume can be added to the mini-bypass device 10 from the venous reservoir bag 140 by opening clamp 149C or from the prime bags 141 by opening clamps 149G and/or 149F and opening clamp 149C. While circulating fluid through the mini-bypass 10 device and A/V conduit 151 using pump 40, air in the system will collect in bubble trap 36 and arterial filter 92 and can be purged to venous reservoir bag 140.

Although a venous reservoir bag is shown, the venous reservoir may be any conventional reservoir device such as a venous reservoir bag, a hardshell venous reservoir or a combined venous cardiotomy reservoir. Additionally, the reservoir may be a venous reservoir capable of vacuum assisted venous drainage. As shown in extracorporeal perfusion system 136, the venous reservoir bag 140 primarily functions as a blood storage reservoir to manage blood volume during the CPB procedure. The venous reservoir bag 140 also serves as a reservoir for any blood/air removed from the bubble trap purge line 142 and/or arterial filter purge line 145 and as a priming reservoir to facilitate autologous priming of the mini-bypass device 10 and the system 136.

Autologous priming is a technique commonly used with mini-bypass systems. The patient's arterial blood pressure provides the force to push blood through the arterial line 139 to the arterial filter 92, while pushing prime solution into the venous reservoir bag 140 through the arterial filter purge line 145. The centrifugal pump 40 draws blood through the venous line 137 and into the mini-bypass device 10, while urging prime solution through the arterial filter purge line 145. Once the patient 52 is on bypass, lowering the prime bags 141 below the height of the reservoir bag 140 can allow prime solution stored in the venous reservoir bag 140 to gravity-drain into the prime bags 141.

At the option of the user, the venous reservoir bag 140 could function as a conventional closed venous reservoir by closing the venous line clamp 149A between the reservoir inlet 100 and outlet 98, unclamping the reservoir inlet and outlet line clamps 149B and 149C, respectively, and allowing gravity or vacuum assisted venous drainage into the venous reservoir bag 140.

In the closed-loop mode, air that may enter system 136 and the mini-bypass device 10 is removed through the bubble trap purge line 142 and arterial filter purge line 145. The bubble trap purge line 142 connects to the top of the bubble trap 36 and to the top of the venous reservoir bag 140. The bubble trap 36 is normally under negative pressure, with roller pump 38 typically removing air/blood through the bubble trap purge line 142. The arterial filter purge line 145 connects to the top of the arterial filter 92 and to the top of the venous reservoir bag 140. Positive pressure generated by the centrifugal pump 40 purges air/blood when the arterial filter purge port 102 is in an open position. The bubble trap purge line 142 and arterial filter purge line 145 each contain a one-way valve to prevent retrograde flow. By opening clamp 149C and draining the reservoir bag 140, blood removed through the bubble trap purge line 142 or the arterial filter purge line 145 may return to the extracorporeal system 136.

Although not shown, a blood gas sampling system, typical of those used in CPB procedures, could be incorporated into the system. An arterial line of the blood gas sampling system would be connected to a port at the top of the arterial filter 92 or to a port on the arterial line 139. The venous line of this sampling system would connect to a port on the venous line 137. Due to the pressure differential between the arterial and venous sides of the system, blood would flow through the sampling system from the arterial to venous side, allowing appropriate blood gas monitoring to be performed as is known in the art.

Although not shown, pressure-monitoring lines typical of those used in cardiopulmonary bypass procedures can also be incorporated into the system. The venous pressure monitoring line would attach to a port on the venous line 137. The arterial pressure monitoring line would attach to a port near the arterial port 134 on the mini-bypass device 10. When connected in this manner, these lines would allow venous and arterial pressures to be monitored, as is well known in the art.

In the extracorporeal perfusion system 136, the prime lines 147 connect to an inlet 104 of the venous reservoir bag 140, which may be a cardiotomy inlet. To prime the device 10 and the extracorporeal system 136, priming fluid must first drain from the prime bags 141 into the venous reservoir bag 140.

The vent or suction line(s) of the extracorporeal perfusion system 136 provide venting or suction from the surgical site and may connect to a separate cardiotomy reservoir (not shown). The cardioplegia line of the extracorporeal perfusion system 136 provides delivery of cardioplegia solution to arrest the heart and may connect to a dedicated blood access port on the oxygenator 90. The gas line is typical of those used in CPB procedures.

Convertible Cardiopulmonary Bypass System Containing a Hardshell Venous Reservoir (FIGS. 3A and 3B)

FIGS. 3A and 3B show a convertible extracorporeal blood perfusion system 11 in accordance with the present invention. FIG. 3A shows system 11 connected in a manual operational mode and FIG. 3B shows system 11 connected to include certain sensors and control devices, to be described hereafter, which automate certain functions of the system. The systems of FIGS. 3A and 3B will be described as incorporating the device 10.

As shown in FIGS. 3A and 3B, the convertible extracorporeal blood perfusion system 11 includes various system components that interconnect in a manner that allows the system 11 to convert quickly and safely between a closed-loop CPB system, with all of the advantages stated above, and a standard system that uses a venous reservoir. The venous reservoir may be any conventional venous reservoir device, such as a venous reservoir bag, a hardshell venous reservoir or a combined venous cardiotomy reservoir. Additionally, the reservoir may be a venous reservoir capable of vacuum assisted venous drainage. Reservoir 16 may be used for the output of the left ventricular vent line and the cardiotomy suction line, if used. The system 111 is shown using a sealed hardshell venous reservoir 16 that is capable of vacuum assisted venous drainage which provides some advantages, including increased venous flow rate as compared to a gravity drainage venous reservoir. If adequate venous drainage can be achieved without vacuum assistance of venous drainage, the blood perfusion system 11 may function as a typical gravity drainage venous reservoir system.

One of the features of the convertible system 11 is that it may be packaged and shipped to the user already connected, so that set up is fast and efficient. Alternatively, the convertible system 11 may be packaged and shipped with some portion of the components and connections included but leaving the user to add additional components and connections as desired.

The convertible cardiopulmonary perfusion system 11 may be set up in either a closed-loop cardiopulmonary bypass system or a standard system that uses a venous reservoir at the beginning of a CPB procedure. The convertible system 11 may be transformed to the other type of system at any time during the CPB procedure. With this convertible extracorporeal blood perfusion system 11, the benefits of closed-loop cardiopulmonary bypass may be achieved with the additional advantages of a readily available venous reservoir 16. A description of components included in the convertible extracorporeal cardiopulmonary perfusion system 11, as shown in FIGS. 3A and 3B, follows. These systems are similar, so common reference numerals will be used to identify common elements.

The integrated cardiopulmonary mini-bypass device 10, as previously described, includes a venous bubble trap 36, a centrifugal pump 40, an oxygenator/heat exchanger 90, and an arterial filter 92. A venous line 12 allows venous flow from a patient 52 into the mini-bypass device 10 either directly, in a closed-loop cardiopulmonary bypass mode, or through the venous reservoir 16, in a gravity or vacuum assisted venous drainage reservoir cardiopulmonary bypass mode. In either cardiopulmonary bypass mode of system 11, the arterial line 14 returns blood to the patient 52 after oxygenation, filtration, temperature regulation, etc. by the mini-bypass device 10.

Device 10 includes a venous bubble trap 36 having an inlet 128 that receives venous blood from a patient 52 through venous line 12. The centrifugal pump 40 that connects between the bubble trap 36 and the oxygenator/heat exchanger 90 creates a negative pressure in the bubble trap 36 and in the venous line 12 to assist in drawing blood from the patient 52. The centrifugal pump 40 draws venous blood through the bubble trap 36, venous bubble trap outlet 37, and tubing 39 into the inlet 41 of centrifugal pump 40. The pump 40 supplies venous blood to an inlet of the oxygenator/heat exchanger 90. The oxygenator/heat exchanger 90 oxygenates the blood and controls the blood temperature. The oxygenated and temperature controlled blood is then supplied from an oxygenator/heat exchanger 90 outlet to an arterial filter 92 inlet. The filtered, oxygenated and temperature controlled blood exits the mini-bypass device 10 at the arterial outlet 134 and returns to the patient 52 via arterial line 14.

The venous bubble trap 36 has a bubble trap purge port 132 that connects to a bubble trap purge line 143 that contains one way valve 30. When air accumulates at the top of bubble trap 36, a roller pump 38 connected to line 143 may be actuated manually by the operator, as shown for the system of FIG. 3A, or automatically, as shown for the system of FIG. 3B, to purge air/blood from the bubble trap 36 to a venous reservoir 16.

The arterial filter 92 has an arterial filter recirculation/purge port 102. Arterial filter purge line 32 connects between the arterial filter recirculation/purge port and a port 17 on the reservoir 16. Positive pressure generated by the centrifugal pump 40 purges air/blood when the arterial filter recirculation/purge port 102 is in an open position.

Blood collected with the air evacuated from the venous bubble trap 36 or arterial filter 92 may be pumped to the venous reservoir 16 (or to a separate cardiotomy reservoir, not shown) and recovered for return to the patient 52. Certain other closed-loop CPB systems transfer all retrieved blood to a waste container and do not recover this blood for return to the patient 52. Any volume lost in such systems must be replaced with crystalloid prime, resulting in increased hemodilution, or with allogeneic blood products.

The A/V bridge 18 allows for recirculation and volume management of a patient's blood while on bypass. The A/V bridge 18 includes an arterial line 94 connected to a venous line 96. A reservoir outlet line 26 connects the venous reservoir 16 to the A/V bridge 18. A reservoir inlet line 28 connects the venous line 12 to the venous reservoir 16. By placement of various clamps on the lines 94, 96 on the A/V bridge 18 and elsewhere in the extracorporeal blood perfusion system 11, as described further herein, the system 11 may be configured in several different ways.

The prime lines 20 function to prime the extracorporeal blood perfusion system 11 from one or more prime solution bags 22 and to assist in volume management. One or more blood storage bags 24 may replace one of the prime solution bags 22 after the system 11 is primed. Blood storage bags 24 are used to retain excess volume to assist in volume management.

Closed-Loop Mode Connection System

FIGS. 3A and 3B illustrate the convertible system 11 in both the closed-loop mode connection system and the standard mode (gravity drainage or vacuum assisted venous drainage connection system. To configure the closed-loop mode connection system, clamp 50 closes the reservoir inlet line 28, clamp 44 closes the arterial line 94 of the A/V bridge 18 and clamp 46 closes the venous line 96 of the A/V bridge 18 so that extracorporeal circulation bypasses the venous reservoir 16. Additionally, clamp 48 as shown in FIG. 3A or clamp 86 as shown in FIG. 3B and clamps 21 on rapid prime lines 20 are closed. Venous line clamp 51 and arterial line clamp 61 are open. In the closed-loop bypass mode, venous blood flows directly from the patient 52 through the venous line 12 and into the mini-bypass device 10, and arterial blood flows directly from the mini-bypass device 10 through the arterial line 14 and back to the patient 52.

In the closed-loop cardiopulmonary bypass connection system, the reservoir 16 is used for volume management and possibly as a cardiotomy reservoir for managing blood vented or suctioned from the cardiotomy field. Because the venous reservoir 16 is inactive in the closed-loop CPB mode, blood volume must be managed differently than when the venous reservoir 16 is an integral, functioning part of the cardiopulmonary bypass system 11. If necessary, blood may be removed from the patient 52 and stored in either the venous reservoir 16 or a blood storage bag 24. Using the pressure generated by the centrifugal pump 40, blood volume is transferred to the venous reservoir by opening the clamp 44 on the arterial line 94 of the A/V bridge 18 and opening the reservoir outlet line clamp 48 as shown in FIG. 3A or clamp 86 as shown in FIG. 3B. Blood volume is transferred to the blood storage bag 24 by opening the clamp 44 on the arterial line 94 of the A/V bridge 18 and opening the clamp 21 to the blood storage bag 24. If necessary, volume may be added to the patient 52 by adding blood, prime solution or other blood/blood-products from the venous reservoir 16, a prime solution bag 22, or a blood storage bag 24. Opening the clamp 46 on the venous line 96 of the A/V bridge 18 and opening the reservoir outlet line clamp 48 as shown in FIG. 3A or clamp 86 as shown in FIG. 3B allows blood to flow from the reservoir 16 into the venous line 96 of the A/V bridge 18, through the mini-bypass device 10 and back to the patient 52 through the arterial line 14. In the same manner, volume may be added from either a prime solution bag 22 or the blood storage bag 24 by opening a clamp 21 and opening the clamp 46 on the venous line 96 of the A/V bridge 18.

Standard Mode Connection System

The system 11 can be configured in the standard mode (gravity drainage or vacuum assisted venous drainage) connection system by opening the reservoir outlet tubing clamp 48 as shown in FIG. 3A or clamp 86 as shown in FIG. 3B, opening the venous clamp 46, opening the reservoir inlet line clamp 50, closing the arterial clamp 44, and closing clamp 51 to close the venous line 124 between the reservoir inlet line 28 and the A/V bridge 18 (and closing both clamps 21 on rapid prime lines 20). With the clamps 44, 46, 48 (or 86), 50, 51 configured in this manner, venous blood flows into the venous reservoir 16, out of the reservoir 16, and into the mini-bypass device 10. A reservoir vent port (not shown) connects to a vacuum source (not shown) to provide vacuum-assisted venous drainage, if necessary. Because closed-loop bypass systems are typically placed close to the patient 52 and use a small diameter (e.g., ⅜ inch (0.95 cm) diameter) venous line to reduce the required system prime volume, the use of vacuum assistance of venous drainage is preferable to achieve adequate venous drainage with this standard connection mode of the system 11.

The venous reservoir 16 becomes a functioning element of the standard mode connection system by configuring clamps 21, 44, 46, 48 (or 86), 50 as described in the previous paragraph. Adequate volume must be available in or be added to the reservoir 16. Additionally, a separate cardiotomy reservoir (not shown) may be part of the convertible extracorporeal cardiopulmonary bypass system 11.

Due to circumstances encountered, e.g. persistent venous air, or at the discretion of the surgical team, the convertible extracorporeal blood perfusion system 11 may easily be converted from closed loop bypass mode to a hardshell gravity or vacuum assisted venous drainage bypass mode during the cardiopulmonary procedure. If the patient 52 is already on bypass when conversion to the standard mode connection system is initiated, centrifugal pump speed 40 is slowed to reduce blood flow rate while maintaining adequate flow and arterial blood pressure. Opening the reservoir outlet line clamp 48 as shown in FIG. 3A or clamp 86 as shown in FIG. 3B, the venous clamp 46, and the reservoir inlet line clamp 50 includes the reservoir 16 in extracorporeal flow. Venous line clamp 51 then closes the venous line 124 between the reservoir inlet line 28 and the A/V bridge 18.

Alternately, the system 11 may be set up in the hardshell gravity drainage or vacuum assisted venous drainage cardiopulmonary bypass mode at the start of the procedure. To configure the standard mode connection system prior to initiation of bypass, the centrifugal pump 40 is stopped and the venous clamp 46 and reservoir outlet line clamp 48 as shown in FIG. 3A or clamp 86 as shown in FIG. 3B are opened. Venous line clamp 51 closes the venous line 124 between the reservoir inlet line 28 and the A/V bridge 18. If vacuum is used, the vacuum level in the reservoir is monitored to achieve adequate venous return. The arterial clamp 44 remains closed while the patient 52 is on standard bypass mode. The centrifugal pump 40 draws blood, filtered and defoamed by the reservoir 16, to cycle through the mini-bypass device 10 and return to the patient 52 via the arterial line 14.

Termination of Cardiopulmonary Bypass

Termination of a cardiopulmonary bypass procedure in either the manual (FIG. 3A) or the automated (FIG. 3B) mode of system 11 proceeds as follows. If using vacuum assisted venous drainage, the reservoir 16 is opened to atmosphere prior to termination of bypass. Gas flow is stopped. The centrifugal pump 40 speed is slowly reduced If the system is on open-loop bypass, the venous line 12 between the patient 52 and the reservoir inlet line 28 is occluded by closing clamp 50 immediately followed by clamping the arterial line by closing clamp 61. If the system is on closed-loop bypass, the arterial line is occluded by closing clamp 61 followed by clamping the venous line by closing clamp 51.

The reservoir outlet line 26 is opened. The arterial and venous clamps 44, 46 are opened. The pump speed is reduced to continue recirculation as required. The heat exchanger continues operation during the recirculation phase. If present, the cardioplegia system connected to the arterial blood access port is occluded.

Manually Operated Convertible Cardiopulmonary Bypass System

FIG. 3A illustrates system 11 configured in manual operation mode. Air that collects in the bubble trap 36 is removed by manually activating roller pump 38. At the same time the centrifugal pump 40 speed is manually decreased to reduce the venous line 12 negative pressure and reduce the air entrainment rate. Reduced centrifugal pump 40 speed also increases the residence time of air in the venous bubble trap 36 for more effective air removal. Blood removed with the air evacuated from the venous bubble trap 36 or is collected in the venous reservoir 16 and recovered for return to the patient 52. This task may be accomplished by manually opening clamp 48 on the reservoir outlet line 26 and clamp 46 on the venous line 96 of the A/V bridge 18, and allowing fluid to drain from the reservoir 16 back into the systemic circulation.

Convertible Cardiopulmonary Bypass System Hardware Systems

FIG. 3B illustrates system 11 in which sensors and control devices manage certain functions of a convertible cardiopulmonary bypass system.

An electric clamp, such as the Stöckert ERC Clamp, may desirably be installed as the clamp 60 on the arterial line 14. The Stöckert ERC is a clamp with a mechanical remote control, a mechanical occlusion mechanism and an individually positionable, instantly accessible control unit. The Stöckert ERC Clamp is available from Sorin Group Deutschland GmbH, Munich, Germany.

An ultrasonic bubble detector, such as the Stöckert Ultrasonic Bubble Detector, may suitably be installed as a venous line bubble detector 82. The Stöckert Ultrasonic Bubble Detector is available from Sorin Group Deutschland GmbH, Munich, Germany. Audible and visual alarms of the ultrasonic bubble detector alert the user to the presence of air bubbles in the venous line, and the roller pump 38 automatically activates for a predetermined time, e.g. about five seconds, to remove detected air from the bubble trap 36. The centrifugal pump 40 reduces speed to reduce the venous line 12 negative pressure and reduce the air entrainment rate. Reduced centrifugal pump 40 speed also increases the residence time of air in the venous bubble trap 36 for more effective air removal.

A centrifugal pump inlet line bubble detector 106 is placed on the tubing between the bubble trap 36 and the centrifugal pump 40 and used in conjunction with the electric clamp 60 on the arterial line 14. The bubble detector 106 may also suitably be an ultrasonic bubble detector. If the bubble detector 106 detects air, the bubble detector 106 automatically causes the clamp 60 to close off the arterial line 14. This detector 106 senses air that may have passed through the bubble trap 36 and may be approaching the centrifugal pump 40. Under these circumstances, massive depriming of the centrifugal pump 40 and passage of large amounts of air further into the system is prevented.

A level sensor 84 is placed on the hardshell venous reservoir 16 and assigned to the clamp 60. If the fluid level in the reservoir 16 falls below the level sensor 84, the clamp 60 automatically closes and prevents flow to the patient. The level sensor 84 may desirably be a Stöckert level sensor. The level sensor 84 can be assigned to the centrifugal pump controller and the electric clamp 60 to stop extracorporeal blood flow and prevent reservoir 16 drainage. If this situation occurs, the user must ensure that the reservoir outlet line 26 is clamped with clamp 86 and then re-establish cardiopulmonary bypass. An adjustable level sensor 84 is an additional feature that would allow maintaining different volume levels, as each procedure may warrant.

In another embodiment, the level sensor 84 is placed on the hardshell venous reservoir 16 and assigned to electric clamp 86. If the fluid level in the reservoir 16 falls below the level sensor 84, the clamp 86 automatically closes and prevents complete drainage of the reservoir 16. Alternately, an electric clamp used with a level sensor can serve to maintain a nearly constant fluid level in the reservoir 16 in the mini-bypass system described herein. This system would be configured such that the clamp 46 on the venous line 96 of the A/V bridge 18 is maintained in the open position. A level sensor would be placed on the reservoir 16 at the desired fluid level. As volume is added to the reservoir 16 and exceeds the height of the level sensor, the electric clamp 86 would open to return this volume to the circuit through reservoir outlet line 26 connected to the venous line 96 of the A/V bridge 18. Once the fluid level falls below the height of the level sensor, the electric clamp 86 would close. This configuration could additionally be used in a conventional CPB system containing a cardiotomy reservoir to maintain a nearly constant fluid level in the cardiotomy reservoir

In another embodiment, the level sensor 84 is placed on the hardshell venous reservoir 16 and assigned to the roller pump 87. The level sensor 84 can be used with a controller and the roller pump 87 to control the automatic and continuous drainage of the venous reservoir. If the fluid level in the reservoir 16 falls below the level sensor 84, the roller pump 87 automatically stops to prevent complete drainage of the reservoir 16.

The above description and the drawings are provided for the purpose of describing embodiments of the invention and are not intended to limit the scope of the invention in any way. It will be apparent to those skilled in the art that various modifications and variations can be made without departing from the spirit or scope of the invention. Thus, it is intended that the present invention covers the modifications and variations of this invention provided they come within the scope of the appended claims and their equivalents.

What is claimed is:

1. A convertible extracorporeal blood perfusion system for receiving venous blood from a patient and for returning oxygenated blood to the patient in a cardiopulmonary bypass procedure comprising:
a closed-loop cardiopulmonary bypass system; and
a circuit for converting between the closed-loop cardiopulmonary bypass system and a cardiopulmonary bypass system containing a venous reservoir,
the circuit for converting comprising a venous reservoir that can be fluidly connected to a venous line from the patient and that can be fluidly connected to the inlet of a first pump, the first pump being part of the closed-loop cardiopulmonary bypass system,
the closed-loop cardiopulmonary bypass system comprising a bubble removal device fluidly connected to the inlet of the first pump.

2. A convertible extracorporeal blood perfusion system of claim 1, further comprising a bubble sensor on the venous line from the patient that is operable to send a signal when gaseous bubbles are detected to a controller that automatically activates a second pump fluidly attached to the bubble removal device in order to purge air from the bubble removal device and venous line.

3. A convertible extracorporeal blood perfusion system of claim 1, further comprising a bubble sensor on a first pump inlet line to the inlet of the first pump that is operable to send a signal when bubbles are detected in the first pump inlet line to a controller that automatically closes a clamp on an arterial line to the patient in order to stop the flow of blood back to the patient.

4. A convertible extracorporeal blood perfusion system of claim 1, wherein the venous reservoir is a soft bag venous reservoir.

5. A convertible extracorporeal blood perfusion system of claim 4, wherein the venous reservoir bag is capable of vacuum assisted venous drainage.

6. A convertible extracorporeal blood perfusion system of claim 1, wherein the venous reservoir is a hardshell venous reservoir.

7. A convertible extracorporeal blood perfusion system of claim 6, wherein the hardshell venous reservoir is capable of vacuum assisted venous drainage.

8. A convertible extracorporeal blood perfusion system of claim 1, wherein the closed-loop cardiopulmonary bypass system comprises an oxygenator and a heat exchanger.

9. A convertible extracorporeal blood perfusion system of claim 8, wherein the bubble removal device, first pump, oxygenator, and heat exchanger are integrated in one unit.

10. A convertible extracorporeal blood perfusion system of claim 1, wherein the closed-loop cardiopulmonary bypass system comprises an oxygenator, a heat exchanger, and an arterial filter.

11. A convertible extracorporeal blood perfusion system of claim 10, wherein the bubble removal device, first pump, oxygenator, heat exchanger, and arterial filter are integrated in one unit.

12. A convertible extracorporeal blood perfusion system of claim 1, further comprising a level sensor that is operable to detect a blood level in the venous reservoir that is below a predetermined level and is operable to send a signal to a controller that automatically stops the flow of blood from the venous reservoir.

13. A convertible extracorporeal blood perfusion system of claim 12, wherein the controller automatically stops the flow of blood from the venous reservoir by closing a clamp on a venous reservoir exit line.

14. A convertible extracorporeal blood perfusion system of claim 12, wherein the controller automatically stops the flow of blood from the venous reservoir by stopping a second pump on a venous reservoir exit line.

15. A convertible extracorporeal blood perfusion system of claim 12, wherein the controller automatically stops the flow of blood from the venous reservoir by closing a clamp on an arterial line to the patient.

16. A convertible extracorporeal blood perfusion system of claim 1, further comprising a level sensor that is operable to detect a blood level in the venous reservoir that is below a predetermined level and is operable to send a signal to a controller that automatically closes a clamp on an arterial line to the patient in order to stop the flow of blood to the patient.

17. A convertible extracorporeal blood perfusion system of claim 1, further comprising a level sensor that is operable to detect a blood level in the venous reservoir that is above a predetermined level and is operable to send a signal to a controller that automatically starts the flow of blood from the venous reservoir.

18. A convertible extracorporeal blood perfusion system of claim 17, wherein the controller automatically starts the flow of blood from the venous reservoir by opening a clamp on a venous reservoir exit line.

19. A convertible extracorporeal blood perfusion system of claim 17, wherein the controller automatically starts the flow of blood from the venous reservoir by starting a second pump on a venous reservoir exit line.

20. A convertible extracorporeal blood perfusion system for receiving venous blood from a patient and for returning oxygenated blood to the patient in a cardiopulmonary bypass procedure comprising:
a closed-loop cardiopulmonary bypass system; and
a circuit for converting between the closed-loop cardiopulmonary bypass system and a cardiopulmonary bypass system containing a venous reservoir,
the circuit for converting comprising a venous reservoir that can be fluidly connected to a venous line from the patient and that can be fluidly connected to the inlet of a first pump, the first pump being part of the closed-loop cardiopulmonary bypass system,
the convertible extracorporeal blood perfusion system comprising a level sensor that is operable to detect a blood level in the venous reservoir that is below a predetermined level and is operable to send a signal to a controller that automatically stops the flow of blood from the venous reservoir.

21. A convertible extracorporeal blood perfusion system of claim 20, wherein the controller automatically stops the flow of blood from the venous reservoir by closing a clamp on a venous reservoir exit line.

22. A convertible extracorporeal blood perfusion system of claim 20, wherein the controller automatically stops the flow of blood from the venous reservoir by stopping a second pump on a venous reservoir exit line.

23. A convertible extracorporeal blood perfusion system for receiving venous blood from a patient and for returning oxygenated blood to the patient in a cardiopulmonary bypass procedure comprising:
a closed-loop cardiopulmonary bypass system; and
a circuit for converting between the closed-loop cardiopulmonary bypass system and a cardiopulmonary bypass system containing a venous reservoir,
the circuit for converting comprising a venous reservoir that can be fluidly connected to a venous line from the patient and that can be fluidly connected to the inlet of a first pump, the first pump being part of the closed-loop cardiopulmonary bypass system,
the convertible extracorporeal blood perfusion system comprising a level sensor that is operable to detect a blood level in the venous reservoir that is below a predetermined level and is operable to send a signal to a controller that automatically closes a clamp on an arterial line to the patient in order to stop the flow of blood from the venous reservoir and the flow of blood to the patient.

24. A method for converting a convertible extracorporeal blood perfusion system for receiving venous blood from a patient and for returning oxygenated blood to the patient in a cardiopulmonary bypass procedure from a closed-loop cardiopulmonary bypass system to a cardiopulmonary bypass system containing a venous reservoir, wherein the convertible extracorporeal blood perfusion system comprises:
a closed-loop cardiopulmonary bypass system; and
a circuit for converting between the closed-loop cardiopulmonary bypass system and a cardiopulmonary bypass system containing a venous reservoir,
the circuit for converting comprising a venous reservoir that can be fluidly connected to a venous line from the patient and that can be fluidly connected to the inlet of a first pump, the first pump being part of the closed-loop cardiopulmonary bypass system,
the closed-loop cardiopulmonary bypass system comprising a bubble removal device fluidly connected to the inlet of the first pump,
the method comprising fluidly connecting the venous reservoir to the venous line from the patient and fluidly connecting the venous reservoir to the inlet of the first pump.

25. A method of claim 24, wherein the convertible extracorporeal blood perfusion system further comprises a bubble sensor on the venous line from the patient that is operable to send a signal when gaseous bubbles are detected to a controller that automatically activates a second pump fluidly attached to the bubble removal device in order to purge air from the bubble removal device and venous line.

26. A method of claim 24, wherein the convertible extracorporeal blood perfusion system further comprises a bubble sensor on a first pump inlet line to the inlet of the first pump that is operable to send a signal when bubbles are detected in the first pump inlet line to a controller that automatically closes a clamp on an arterial line to the patient in order to stop the flow of blood back to the patient.

27. A method of claim 24, wherein the venous reservoir is a soft bag venous reservoir.

28. A method of claim 27, wherein the venous reservoir bag is capable of vacuum assisted venous drainage.

29. A method of claim 24, wherein the venous reservoir is a hardshell venous reservoir.

30. A method of claim 29, wherein the hardshell venous reservoir is capable of vacuum assisted venous drainage.

31. A method of claim 24, wherein the closed-loop cardiopulmonary bypass system comprises an oxygenator and a heat exchanger.

32. A method of claim 31, wherein the bubble removal device, first pump, oxygenator, and heat exchanger are integrated in one unit.

33. A method of claim 24, wherein the closed-loop cardiopulmonary bypass system comprises an oxygenator, a heat exchanger, and an arterial filter.

34. A method of claim 33, wherein the bubble removal device, first pump, oxygenator, heat exchanger, and arterial filter are integrated in one unit.

35. A method of claim 24, wherein the convertible extracorporeal blood perfusion system comprises a level sensor that is operable to detect a blood level in the venous reservoir that is below a predetermined level and is operable to send a signal to a controller that automatically stops the flow of blood from the venous reservoir.

36. A method of claim 35, wherein the controller automatically stops the flow of blood from the venous reservoir by closing a clamp on a venous reservoir exit line.

37. A method of claim 35, wherein the controller automatically stops the flow of blood from the venous reservoir by stopping a second pump on a venous reservoir exit line.

38. A method of claim 35, wherein the controller automatically stops the flow of blood from the venous reservoir by closing a clamp on an arterial line to the patient.

39. A method of claim 24, wherein the convertible extracorporeal blood perfusion system comprises a level sensor that is operable to detect a blood level in the venous reservoir that is below a predetermined level and is operable to send a signal to a controller that automatically closes a clamp on an arterial line to the patient in order to stop the flow of blood to the patient.

40. A method of claim 24, wherein the convertible extracorporeal blood perfusion system comprises a level sensor that is operable to detect a blood level in the venous reservoir that is above a predetermined level and is operable to send a signal to a controller that automatically starts the flow of blood from the venous reservoir.

41. A method of claim 40, wherein the controller automatically starts the flow of blood from the venous reservoir by opening a clamp on a venous reservoir exit line.

42. A method of claim 40, wherein the controller automatically starts the flow of blood from the venous reservoir by starting a second pump on a venous reservoir exit line.
